(19) United States
(12) Patent Application Publication
(10) Pub. No.: US 2012/0179051 A1
(43) Pub. Date: Jul. 12, 2012

(54) APPARATUS AND COMPUTER PROGRAM FOR DETERMINING A PATIENT'S VOLEMIC STATUS REPRESENTED BY CARDIOPULMONARY BLOOD VOLUME

(75) Inventors: Ulrich Pfeiffer, Munich (DE); Frederic Michard, Denens (CH); Reinhold Knoll, Munich (DE)

(73) Assignee: Edwards Lifesciences IPRM AG, Irvine, CA (US)

(21) Appl. No.: 13/315,084

(22) Filed: Dec. 8, 2011

Related U.S. Application Data
(63) Continuation of application No. 11/820,785, filed on Jun. 20, 2007, now abandoned.

(30) Foreign Application Priority Data
Jun. 21, 2006 (DE) 102006028533.6

(51) Int. Cl. A61B 5/0205; A61B 5/021

(52) U.S. Cl. 600/484; 600/485

(57) ABSTRACT

An apparatus for determining a patient's volemic status can make use of a physiological heart-lung interaction during spontaneous breathing or mechanical ventilation. Further, a computer program for determining the patient's volemic status has instructions for carrying out the steps of generating data of a physiological heart-lung interaction during spontaneous breathing or mechanical ventilation, and determining the patient's volemic status when making use of the data of the physiological heart-lung interaction, when run on a computer.

This application is a continuation of U.S. patent application Ser. No. 11/820,785, filed on Jun. 20, 2007 and claims the benefit of German Application No. DE 10 2006 028 533.6, filed on Jun. 21, 2006 and hereby incorporated by reference herein.

The invention relates to an apparatus and a computer program for determining a patient's volemic status represented by cardiopulmonary blood volume CPBV mainly used in fluid management.

### BACKGROUND OF THE INVENTION

It is generally known that in the critical-care diagnosis and treatment of critically ill patients, thoracic blood volumes, i.e. intrathoracic blood volume, right and left heart end-diastolic volumes, are important characteristics for monitoring the patient's state of health and for fluid management of such a patient.

According to prior art the thoracic blood volumes can be determined by using a dilution measurement. A bolus of an indicator defined by a predetermined quantity of the indicator is rapidly injected central-venously into the patient's superior vena cava, and the indicator concentration response is measured at a downstream location of the patient's systemic circulation downstream after cardio-pulmonary passage in the arterial system as close as possible to the outflow of the left ventricle. Based on the indicator concentration response measurement versus time the dilution curve is generated.

During at least one cardiopulmonary circulation, the indicator mainly remains in the intravascular space. E.g. indocyanine green, Evan's blue, or hypertonic saline indicator can be used as intravascular indicator.

The cardiopulmonary blood volume CPBV is represented by the volume of distribution during cardiopulmonary passage which is defined by the multiplication of the cardiac output CO and the mean transit time TT of the respective intravascular indicator, i.e.

CPBV=CO*TT.

It is common to determine the cardiac output CO and the mean transit time TT from the dilution curve. Also, it is known to obtain the cardiac output from any method, which simultaneously displays the cardiac output CO, e.g. echocardiography, transthoracic electrical bioimpedance, or continuous heating right heart catheter, or CO2-rebreathing.

The cardiopulmonary blood volume CPBV which is calculated with above equation consists of the largest accessible distribution volumes for the indicator, which are the sum of the end-diastolic volumes of the right atrium, the right ventricle, the maximum of the pulmonary blood volume during several ventilation cycles within the measurement period, the left atrial and the left ventricular end-diastolic volume, and a smaller portion of aortic blood volume, which is more or less constant.

Another known method for determining the cardiopulmonary blood volume CPBV is a variant of the above method, wherein a single indicator is used. By using this single indicator transpulmonary thermodilution technique, only the sum of the atrial and ventricular end-diastolic blood volumes (i.e. global end-diastolic volume) is exactly measured, whereas the pulmonary blood volume is estimated by assuming that it behaves more or less proportional to the global end-diastolic volume.

When using the known indicator dilution techniques for determining the cardiopulmonary blood volume CPBV, the determination can be done not continuously, but discontinuously, not automatically, but the determination requires user interaction and is labour and cost intensive.

### SUMMARY OF THE INVENTION

It is an object of the invention to provide an apparatus and a computer program for determining a patient's volemic status represented by cardiopulmonary blood volume CPBV, wherein the determination is continuous and easy to achieve.

The present invention provides an apparatus for determining a patient's volemic status, adapted to make use of a physiological heart-lung interaction during spontaneous breathing or mechanical ventilation. Further, the present invention provides a computer program for determining a patient's volemic status, having instructions adapted to carry out the steps of generating data of the physiological heart-lung interaction during spontaneous breathing or mechanical ventilation, and determining the patient's volemic status when making use of the data of the physiological heart-lung interaction, when run on a computer.

Due to the fact that using the physiological heart-lung interaction during spontaneous breathing or mechanical ventilation for determining the cardiopulmonary blood volume CPBV, the determination can be done continuously and automatically without the user's interaction. Therefore, the inventive apparatus and the inventive computer program can perform a less labour intensive and less cost intensive determination of a patient's volemic status.

Preferably, the apparatus is adapted to provide an envelope of the arterial pulse pressure and is capable to determine the physiological heart-lung interaction during mechanical ventilation or spontaneous breathing by making use of the envelope of the arterial pulse pressure.

Furthermore, it is preferred that the computer program has instructions adapted to carry out the steps of providing an envelope of the arterial pulse pressure, and determining the physiological heart-lung interaction by making use of the envelope of the arterial pulse pressure.

The apparatus is preferred to be capable to derive the expiratory cardiopulmonary blood volume CPBVex by making use of the equation

CPBVex=CO*TTcp,ex,

wherein CO is the cardiac output and TTcp,ex is the cardiopulmonary transit time of blood in the hemodynamic status of expiration being derived from the envelope of the arterial pulse pressure. Also, the computer program is preferred to have instructions adapted to carry out the step of deriving the expiratory cardiopulmonary blood volume CPBVex by making use of above equation.

It is preferred that the apparatus is capable to derive the inspiratory left heart volume LHVin by making use of the equation

LHVin=CO*TTlh,in,

wherein CO is the cardiac output and TTlh,in is the inspiratory transit time of blood through the left heart being derived from the envelope of the arterial pulse pressure. Also, the computer program is preferred to have instructions adapted to carry out the step of deriving the inspiratory left heart volume LHVin by making use of above equation.

Alternatively, it is preferred that the apparatus and the instructions of the computer program are adapted to be capable to derive a middle expiratory cardiopulmonary blood volume CPBV by making use of the equation

CPBV=CO*TTcp,

wherein CO is the cardiac output and TTcp is middle cardiopulmonary transit time ranging between the cardiopulmonary transit time of blood TTcp,ex in the hemodynamic status of expiration, and the inspiratory transit time TTlh,in of blood through the left heart, both being derived from the envelope of the arterial pulse pressure.

Preferably the apparatus and the computer program are capable to derive the cardiopulmonary transit time of blood in the hemodynamic status of expiration TTcp,ex by making use of the equation

TTcp,ex=t(B)−t(I−E),

wherein t(I−E) is the time point of end-inspiration and start of expiration, and t(B) is the time point where the envelope of arterial pressure reaches the same level as at the time point of end-expiration and start of inspiration.

Preferably the apparatus and the computer program are capable to derive the inspiratory transit time TTlh,in by making use of the equation

TTlh,in=t(E−I)−t(A),

wherein t(E−I) is the time point of end-expiration and start of inspiration, and t(A) is the time point where the envelope of arterial pressure starts to rise.

Preferably the apparatus is capable to obtain the cardiac output CO from a continuous real time cardiac output measurement method like e.g. arterial pulse contour analysis, esophageal Doppler, transthoracic or esophageal echo Doppler, transthoracic or esophageal electrical Bioimpedance. It is also preferred that the computer program has instructions being adapted to carry out the step of obtaining the cardiac output from above method.

Further, preferably the apparatus is capable to initially check the equilibrium in the cardiopulmonary vascular system by a single extended breathing cycle in investigating as to whether a constant plateau of pulse pressure for expiration is reached in order to necessarily adjust the breathing cycle to a degree with approximate equilibrium. Also it is preferred that the computer program has instructions adapted to carry out the step of above initially checking.

Preferably the apparatus is capable and preferably the computer program has instructions adapted to apply the checking of equilibrium in a pressure controlled ventilation mode or in a volume controlled ventilation mode.

Alternatively, it is preferred that the apparatus is capable to use prolonged step changes of the level of Positive End-Expiratory Pressure (PEEP), and alternatively, it is preferred that the computer program has instructions adapted to carry out the step of using prolonged step changes of the level of Positive End-Expiratory Pressure (PEEP).

As a further alternative, it is preferred that the apparatus is capable to use prolonged step changes of the level of Positive End-Expiratory Pressure PEEP by breathing on three different mean airway pressure (MPaw) levels, and alternatively, it is preferred that the computer program has instructions adapted to carry out the step of using prolonged step changes of the level of Positive End-Expiratory Pressure PEEP by breathing on three different mean airway pressure (MPaw) levels.

Preferably, the apparatus is adapted to compose a phase of low PEEP level PEEP 1, a phase of high PEEP level PEEP 3, and a phase of intermediate PEEP level PEEP 2. Preferably the computer program has instructions adapted to carry out such composing.

It is preferred that the apparatus is adapted to compose the phase of intermediate PEEP level PEEP 2 corresponding to the mean airway pressure Paw mean before a testing phase PEEP 2. Preferably the computer program has instructions adapted to carry out such composing.

Preferably the apparatus is capable to derive the mean cardiopulmonary blood volume CPBVmean by making use of the equation

CPBVmean=COmean*TTcp mean,

wherein COmean is the mean cardiac output CO in the mean positive airway pressure phase after stabilization and TTcp mean is the mean cardiopulmonary transit time of blood being derived from the envelope of the arterial pulse pressure. It is also preferred that the computer program has instructions adapted to carry out the step of deriving the mean cardiopulmonary blood volume CPBVmean by making use of such equation.

Preferably the apparatus is capable to derive the mean cardiopulmonary transit time of blood TTcp mean by making use of the equations

TTcp mean=t(D)−t(3−2),

or

TTcp mean=t(F)−t(1−2),

wherein t(3−2) is the moment of change from the highest level of PEEP or MPaw PEEP 3 to mean positive airway pressure level or the intermediate level of PEEP or MPaw PEEP 2, t(1−2) is the moment of change from the lowest level of PEEP or MPaw PEEP 1 to average positive airway pressure level or the intermediate level of PEEP or MPaw PEEP 2, t(D) is the time point where the envelope of arterial pulse pressure curve has adapted to low PEEP level PEEP 1 or MPaw level, and t(F) is the time point where the envelope of arterial pulse pressure curve has adapted to PEEP or MPaw intermediate level PEEP 2.

The apparatus is preferred to be capable and the computer program has preferred instructions being adapted to derive the mean left heart volume LHVmean by making use of the equation

LHVmean=COmean*TTlh,mean,

wherein COmean is the mean cardiac output CO in the mean positive airway pressure phase after stabilization and TTlh,mean is the mean transit time of blood being derived from the envelope of the arterial pulse pressure. The computer program preferably has instructions adapted to carry out the step of deriving the mean left heart volume LHVmean by making use of such equation.

Further, preferably the apparatus is capable to derive the mean transit time of blood TTlh,mean by making use of the equation

TTlh,mean=t(1−2)−t(E)

wherein t(E) is the time point where the envelope curve of arterial pulse pressure starts to rise. Preferably the computer program has instructions being adapted to carry out such determination.

Preferably the apparatus is capable and preferably the computer program has instructions adapted to derive the slope of Starling curve during the triphasic positive airway pressure (TriPAP) ventilation/respiration mode for the total heart by making use of the difference quotient built from

delta SV over delta CPBV on 3 PEEP or MPaw levels, and
calculating the slope of the curve fitted through these 3 points,
and similarly for the left heart by making use of the difference quotient built from
delta SV over delta LHV on 3 PEEP or MPaw levels.

### BRIEF DESCRIPTION OF THE DRAWINGS

In the following the invention is explained on the basis of preferred embodiments with reference to the drawings. In the drawings:

FIG. 1 is a first diagram showing a curve of the airway pressure, a bar graph of the arterial pressure, and an envelope of the pulse pressure,

FIG. 2 is a second diagram showing a curve of the airway pressure, a bar graph of the arterial pressure, and an envelope of the pulse pressure,

FIG. 3 is a third diagram showing a curve of the airway pressure, a bar graph of the arterial pressure, and an envelope of the pulse pressure, and

FIG. 4 shows schematically an apparatus for producing the diagrams of FIGS. 1 to 3.

### DETAILED DESCRIPTION

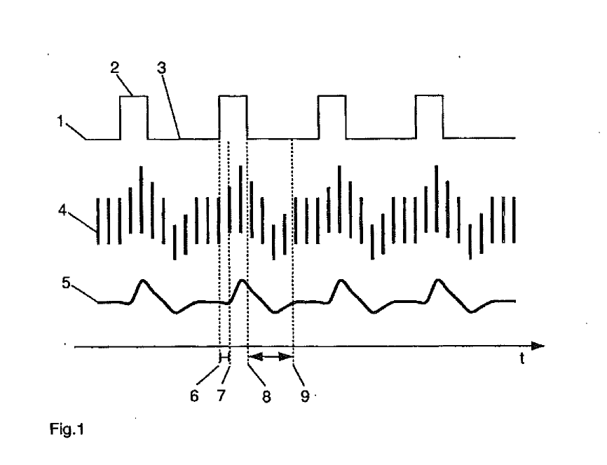

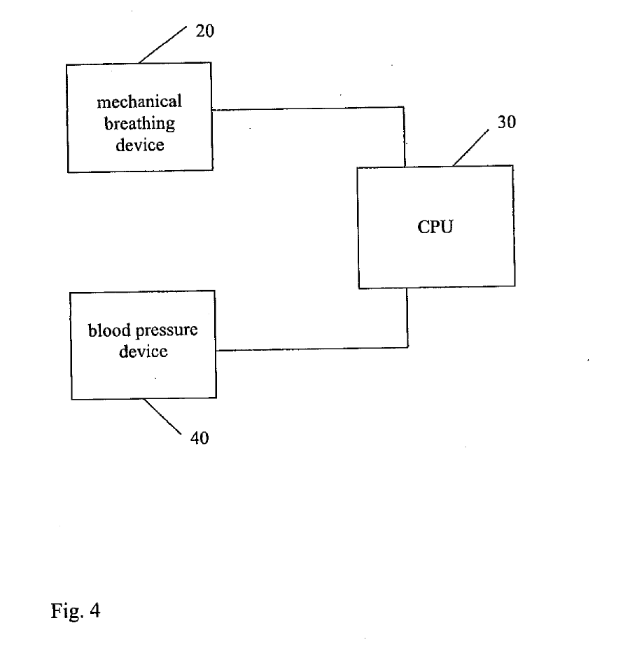

One embodiment of an inventive apparatus as shown in FIG. 4 is adapted to provide a diagram shown in FIG. 1, with a mechanical breathing device 40 (or breathing monitor), a blood pressure device 20 and a computer or processor 30 being connected to the blood pressure device 20 and mechanical breathing device 40. In the diagram a curve of the airway pressure 1 providing successive inspiration phases 2 and expiration phases 3 is shown and may be derived from breathing device 40, wherein during the inspiration phases 2 the airway pressure is higher than during the expiration phases 3. Further, in the diagram an arterial pressure diagram 4 is shown, Wherein for each heart beat a vertical bar reaching from diastolic pressure (minimum pressure) up to systolic pressure (maximum pressure) is plotted. Additionally, in the diagram an envelope of the arterial pulse pressure 5 is shown, which is defined as being the difference between the systolic pressure and the diastolic pressure according to the arterial pressure diagram 4. Both diagrams 4 and 5 may be derived from blood pressure device 20.

The mammalian thorax can be regarded as a chamber with a variable volume. The chamber is composed of the partial volumes of the heart, the lungs, the large extra cardiac vessels, connective tissue and the esophagus. The thoracic volume changes regularly with breathing or mechanical ventilation. Under pathophysiological conditions, it may vary due to increased abdominal pressure, and also due to external pressure, for example during diving, etc.

Looking at a variable thoracic volume in terms of time, it contains partial volumes which change very rapidly, for example within seconds, in the course of a breathing or ventilation cycle, as does the gas volume inside the lungs and the blood volume inside large vessels and inside the heart, and partial volumes which change over longer time periods, such as the functional residual volume of the lungs due to therapeutic intervention, e.g. application of positive end-expiratory pressure, an increase in extravascular lung water (e.g., when pulmonary edema is formed), and an increase in pathological partial volumes (e.g., as in case of hematothorax, pneumothorax or pleural effusion).

In the case of spontaneous breathing, the inhaled air enters the lungs due to the negative intrathoracic pressure ITP which is produced by the thoracic intercostal musculature and the diaphragm. However, the venous blood flow into the chest region, often called venous return, is facilitated during spontaneous inhalation as well. During exhalation in spontaneous breathing, the intrathoracic pressure becomes positive again, which causes gas to leave the lungs, since the pressure within the lungs exceeds atmospheric pressure, while the venous return is slowed down. Exactly the same happens during mechanical respiration when spontaneous breathing is simulated by means of a chamber respirator in the form of an iron lung.

During mechanical respiration, in particular positive-pressure ventilation, inhaling is accomplished by producing a positive gas pressure in the airways by the mechanical ventilator outside the lungs. Respiratory gas enters the lungs because the airway pressure inside the lungs is lower. Gas enters the lungs until the airway pressure in the external airways and the airway pressure in the lungs and internal airways reach equilibrium. During this inhalation process, the lungs are inflated, which increases the intrathoracic pressure, and the large blood vessels (intrathoracic vena cava and aorta) and the heart itself are compressed.

From the physiological point of view, this means that venous blood flow to the right heart, i.e. venous return, is reduced. Exhalation occurs due to the retractive force of the thoracic walls, the diaphragm, and the lungs themselves and, to a lower degree, due to the weight of the thoracic wall itself, whereby the intrathoracic pressure ITP drops again while the venous return increases.

The apparatus is adapted to use interactions between the heart and the lungs during breathing and in particular during mechanical ventilation. Therefore, the above described changes in venous return during spontaneous breathing as well as during mechanical positive pressure ventilation do have a direct effect on the cardiac filling and—via the Frank Starling mechanism—on the ventricular output, i.e. the cardiac stroke volume. The Starling mechanism describes a relationship between the diastolic cardiac filling volume and the systolic cardiac stroke volume to that effect that the more a cardiac chamber is filled in the diastolic phase, the greater is the output of systolic cardiac stroke volume.

In timely manner the following occurs in the cardio-pulmonary vascular system during mechanical positive pressure ventilation with inflation and deflation of the lungs:
With beginning of inflation of the lungs venous return and right ventricular filling and stroke output decrease, pulmonary blood volume, i.e. blood in the lungs, is squeezed out of the lungs and for a short period of time increases left ventricular filling and stroke volume output.
When the blood in the lungs is completely squeezed out (at end of inflation) also left ventricular filling and consequently left ventricular stroke volume output decrease.
With beginning of deflation (i.e. expiration) venous return to the right ventricle and right ventricular stroke volume output start to increase again.
When the blood volume in the lungs has come up to its new equilibrium also left ventricular filling and stroke volume output have increased to their level right before the start of the mechanical breath.

When the ventilation rate, each of the durations of the inspiration phases 2, and each of the durations of the expiration phases 3 are long enough, equilibrium in the cardiopulmonary vascular system can occur in each of the inspiration phases and the expiration phases.

Referring to FIG. 1, the apparatus using computer 30 is capable to calculate the cardiopulmonary transit time of blood TTcp,ex in the hemodynamic status of expiration from the course of the arterial pulse pressure wave envelope 5 by using the equation

TTcp,ex=t(B)−t(I−E),

wherein t(I−E) is defined as being the time point 8 of end-inspiration and t(B) is defined as being the time point 9 where the envelope of arterial pulse pressure 5 reaches the same level as at as at the time point 6 of end-expiration and start of inspiration.

Under the same conditions and in a similar way the apparatus is adapted to calculate the inspiratory transit time TTlh,in of blood through the left heart (i.e. left atrium and ventricle) by using the equation

TTlh,in=t(E−I)−t(A),

wherein t(E−I) is the time point 6 of end-expiration and t(A) is the time point 7 where the envelope curve of arterial pulse pressure 5 starts to rise.

Further, the apparatus is adapted to multiply the respective transit time by the respective mean cardiac output CO during the respective time period in which the respective transit time TT has been determined. Therefore, the apparatus is adapted to calculate the expiratory cardiopulmonary blood volume CPBVex and the inspiratory left heart blood volume LHVin using the equations

CPBVex=CO*TTcp,ex,

and

LHVin=CO*TTlh,in,

respectively.

The apparatus is adapted to obtain the cardiac output CO from any continuous real time cardiac output measurement method like arterial pulse contour analysis, esophageal Doppler, transthoracic or esophageal echo Doppler, transthoracic or esophageal electrical Bioimpedance or others.

Preferably the apparatus is adapted to initially check the equilibrium in the cardiopulmonary vascular system by a single extended breathing cycle. Thereby a constant plateau of pulse pressure for expiration must be reached. This principle could be applied in a pressure controlled ventilation mode (as shown in FIG. 1), or in a volume controlled ventilation mode (not shown in FIG. 1). Afterwards the breathing cycle is adjusted to a degree where equilibrium is approximately reached.

Alternatively, the apparatus is adapted to calculate the cross correlation between airway pressure and pulse pressure even in spontaneous breathing patients with irregular breathing patterns. The delay of the maximum peak of the cross correlation function would provide a middle cardiopulmonary transit time TTcp which ranges in between TTcp,ex and TTlh,in. The middle transit time TTcp multiplied with the mean cardiac output CO is to be used to estimate a middle cardiopulmonary blood volume, i.e.

CPBV=CO*TTcp.

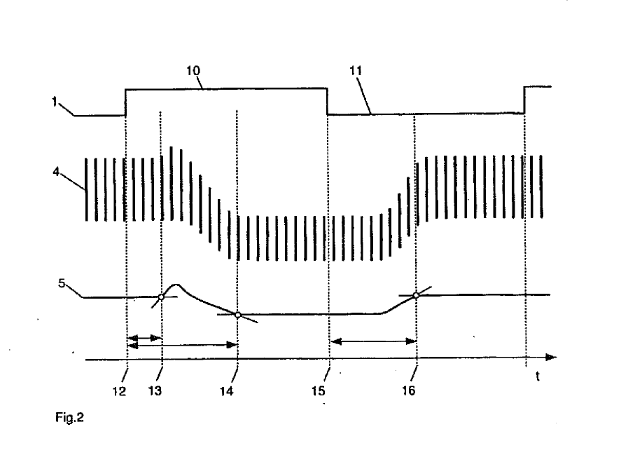

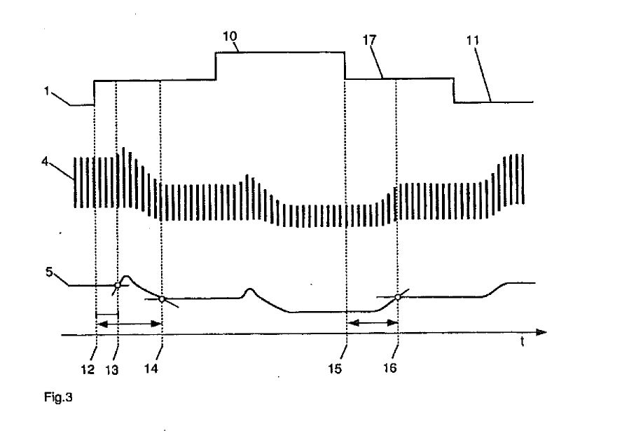

The apparatus is further improved by being adapted to use prolonged step changes of the level of Positive End-Expiratory Pressure PEEP or by breathing on three different mean airway pressure levels MPaw, instead of short term phasic changes in airway pressure during a single mechanical breath according to the diagram shown in FIG. 1. Therefore, the apparatus is adapted to provide diagrams as shown in FIGS. 2 and 3.

In the diagram shown in FIG. 2 a curve of the airway pressure 1 providing the successive high PEEP or MPaw levels (PEEP 3) 10 and the low PEEP or MPaw levels (PEEP 1) 11 is shown. Further, in the diagram shown in FIG. 3 a curve of the airway pressure 1 providing the successive high PEEP or MPaw levels (PEEP 3) 10, middle PEEP or MPaw levels (PEEP 2) 17 and the low PEEP or MPaw levels (PEEP 1) 11 is shown.

Furthermore, in the diagrams shown in FIGS. 2 and 3, respectively, an arterial pressure diagram 4 is shown, wherein for each heart beat a vertical bar reaching from diastolic pressure (minimum pressure) up to systolic pressure (maximum pressure) is plotted. Additionally, in the diagrams, respectively, an envelope of the arterial pulse pressure 5 is shown, which is defined as being the difference between the systolic pressure and the diastolic pressure according to the arterial pressure diagram 4.

Any change in the PEEP level or MPaw level causes a simultaneous corresponding change of intrathoracic pressure which compresses the low pressure capacitance blood vessel system in the chest and changes venous return, right ventricular and left ventricular preload, and hence stroke volume output. This results in the PEEP/MPaw level analogous phase shifted swings in the envelope of the arterial pulse pressure wave 5.

The PEEP or MPaw procedure is composed of a maneuver similar to bi-phasic PEEP or MPaw which originally has been introduced as BIPAP (Biphasic positive airway pressure; Benzer & Baum 1989); i.e. a phase of low PEEP 11 (PEEP 1) and a phase of high PEEP 10 (PEEP 3), and a third phase on the PEEP level 17 which corresponds to the mean airway pressure (Paw mean) before the testing phase (PEEP 2) (see FIG. 3). Hence a tri-phasic mean airway pressure pattern results which will be named “TriPAP”.

When producing the TriPAP ventilation pattern, it needs to be set on a ventilator either manually or by remote control through the hemodynamic monitor, and the information on the pattern, timing and PEEP or MPaw levels can either be fed into the hemodynamic monitor, which does all calculations, directly via cable or via wireless connection.

Alternatively the information can be obtained from feeding airway pressure or a surrogate for intrathoracic pressure like esophageal pressure or an electrical bioimpedance respiration signal directly into the hemodynamic monitor. Further, any step change in PEEP or MPaw level is also simultaneously and immediately reflected in a corresponding change of central venous pressure, hence this information can also be obtained from direct analysis of central venous pressure in the hemodynamic monitor itself.

The apparatus is adapted to use the TriPAP mode as normal either controlled ventilation mode or during spontaneous ventilation as well with the later being superimposed, Once this ventilation TriPAP is set as continuous ventilation/respiration mode, the hemodynamic test can be automatically repeated continuously as well.

The measurement of the respective transit time for calculation of cardiopulmonary blood volume CPBV and the inspiratory left heart volume LHEV is of clinical interest mainly during Paw mean conditions. The respective PEEP or MPaw levels are kept until stabilization of the arterial pulse pressure curve envelope occurs, which is observed when the cardiopulmonary blood volume CPBV has adapted to the new PEEP or MPaw level, hence some seconds later than the cardiopulmonary transit time TTcp.

The apparatus is adapted to calculate the cardiopulmonary transit time TTcp in the phases starting with switching to mean airway pressure coming either down from the highest level 10 of PEEP or MPaw (at time 15 t(3−2)) or coming up from the lowest level 11 of PEEP or MPaw (at time 12 t(1−2)) in two ways:
from the beginning of the step change of PEEP or MPaw until the crossing point of the line of backward extrapolation of the pulse pressure envelope with the tangent at the steepest point on the downslope or upslope of the pulse pressure envelope, or
from the beginning of the step change of PEEP or MPaw until the first derivative of the pulse pressure envelope reaches zero again after the maximum (time from 15 t(3−2) to 16 t(D)) or reaches zero again after the minimum (time 12 t(1−2) to 14 t(F)).

This results in the equations

TTcp mean=t(D)−t(3−2),

and

TTcp mean=t(F)−t(1−2),

wherein t(3−2) is the moment 15 of change from the highest level 10 of PEEP or MPaw (PEEP 3) to mean positive airway pressure level or the intermediate level 17 of PEEP or MPaw (PEEP 2), t(1−2) is the moment 12 of change from the lowest level 11 of PEEP or MPaw (PEEP 1) to average positive airway pressure level or the intermediate level 17 of PEEP or MPaw (PEEP 2), t(D) is the time point 16 where the envelope of arterial pulse pressure curve 5 has adapted to intermediate PEEP or MPaw level 17, and t(F) is the time point 14 where the envelope of arterial pulse pressure curve 5 has adapted to intermediate PEEP or MPaw level 17.

Using above mean transit time TTcp mean, the apparatus is adapted to calculate the mean cardiopulmonary blood volume CPBVmean by solving the equation

CPBVmean=COmean*TTcp mean,

wherein COmean is the mean cardiac output CO in the mean positive airway pressure phase after stabilization.

The mean left heart blood volume LHVmean can be calculated only during step changes where blood is squeezed out of the lungs, which happens only with an increase of intrathoracic pressure with increasing PEEP or MPaw. Thus, the apparatus is capable of calculating the mean left heart blood volume LHVmean by solving the equation

LHVmean=COmean*TTlh,mean,

wherein the apparatus is adapted to calculate the mean transit time TTlh,mean by solving the equation

TTlh,mean=t(1−2)−t(E)

wherein t(E) is the time point 13 where envelope curve of arterial pulse pressure 5 starts to rise.

In conventional ventilators BIPAP ventilation/respiration is a common mode. Since this mode has got only two PEEP or MPaw levels, a high PEEP or MPaw level 10 (PEEP 3) and a low PEEP or MPaw level 11 (PEEP 1), a respective mean airway pressure cannot be set. In this mode the mean cardiopulmonary blood volume CPBVmean can be continuously estimated as floating average from (CPBVPEEP1+CPBVPEEP3)/2 or (CPBVMPaw1+CPBVMPaw3)/2 in the same way as described above.

With regard to the left heart blood volume LHV, only LHVPEEP3 is obtained directly. However, assuming a parallel change of the cardiopulmonary blood volume CPBV and the left heart blood volume LHV during changes in PEEP or MPaw levels, the mean cardiopulmonary blood volume LHVmean can be estimated by multiplying LHVPEEP3 with the ratio CPBVmean/CPBVPEEP3.

Further, the apparatus is adapted to derive the parameters GEF and LHEF by making use of the equations

GEF=4*SV/CPBV K,

and

LHEF=2*SV/LHV*K,

respectively. Where GEF is the Global Ejection Fraction, LHEF is the Left Heart Ejection Fraction. Coefficient K is an empirical correction factors to adjust GEF and LHEF to the values obtained from transpulmonary double indicator thermal dye dilution measurements.

Further, the apparatus is adapted to derive the slope of Starling curve during TriPAP for the total heart by making use of the difference quotient built from
delta SV over delta CPBV on 3 PEEP or MPaw levels, and
   for the left heart by making use of the difference quotient built from
delta SV over delta LHV on 3 PEEP or MPaw levels.

Alternatively all calculations could be performed also with systolic arterial pressure or mean arterial pressure instead of pulse pressure. Alternatively in all calculations the airway pressure could be replaced by the central venous pressure or a surrogate of intrathoracic pressure like esophageal pressure or an electrical bioimpedance respiration signal.

Taking above mentioned and described modelling into account, a process for determining a patient's volemic status comprises the steps of:
Generating data of a physiological heart-lung interaction during spontaneous breathing or mechanical ventilation.
Determining the patient's volemic status when making use of the data of the physiological heart-lung interaction.
Providing an envelope of the arterial pulse pressure 5.
Determining the physiological heart-lung interaction by making use of the envelope of the arterial pulse pressure 5.
Deriving the expiratory cardiopulmonary blood volume CPBVex by making use of the equation

CPBVex=CO*TTcp,ex,

wherein CO is the cardiac output and TTcp,ex is the cardiopulmonary transit time of blood in the hemodynamic status of expiration being derived from the envelope of the arterial pulse pressure 5.
Deriving the inspiratory left heart volume LHVin by making use of the equation

LHVin=CO*TTlh,in,

wherein CO is the cardiac output and TTih,in is the inspiratory transit time of blood through the left heart being derived from the envelope of the arterial pulse pressure 5.
Deriving the cardiopulmonary transit time of blood in the hemodynamic status of expiration TTcp,ex by making use of the equation

TTcp,ex=t(B)−t(I−E),

wherein t(I−E) is the time point 8 of end-inspiration and start of expiration, and t(B) is the time point 9 where the envelope of arterial pressure 5 reaches the same level as at the time point 6 of end-expiration and start of inspiration.
Deriving the inspiratory transit time TTlh,in by making use of the equation

TTlh,in=t(E−I)−t(A),

wherein t(E−I) is the time point 6 of end-expiration and start of inspiration, and t(A) is the time point 7 where the envelope of arterial pressure 5 starts to rise.
Obtaining the cardiac output CO from a continuous real time cardiac output measurement method like arterial pulse contour analysis, esophageal Doppler, transthoracic or esophageal echo Doppler, transthoracic or esophageal electrical Bioimpedance.
Initially checking the equilibrium in the cardiopulmonary vascular system by a single extended breathing cycle in investigating as to whether a constant plateau of pulse pressure for expiration is reached in order to necessarily adjust the breathing cycle to a degree with approximate equilibrium.
Applying the checking of equilibrium in a pressure controlled ventilation mode or in a volume controlled ventilation mode.

Alternatively, a process for determining a patient's volemic status comprises the step of:
Deriving a middle expiratory cardiopulmonary blood volume CPBV by making use of the equation

CPBV=CO*TTcp,

wherein CO is the cardiac output and TTcp is middle cardiopulmonary transit time ranging between the cardiopulmonary transit time of blood TTcp,ex in the hemodynamic status of expiration, and the inspiratory transit time TTlh,in of blood through the left heart, both being derived from the envelope of the arterial pulse pressure 5.

Alternatively, a process for determining a patient's volemic status comprises the step of:
Using prolonged step changes of the level of Positive End-Expiratory Pressure PEEP, or using prolonged step changes of the level of Positive End-Expiratory Pressure PEEP by breathing on three different mean airway pressure levels MPaw.
Composing a phase of low PEEP level (PEEP 1) 11, a phase of high PEEP level (PEEP 3) 10, and a phase of intermediate PEEP level (PEEP 2) 17.
Composing the phase of intermediate PEEP level (PEEP 2) 17 corresponding to the mean airway pressure Paw mean before a testing phase PEEP 2.
Deriving the mean cardiopulmonary blood volume CPBVmean by making use of the equation

CPBVmean=COmean*TTcp mean,

wherein COmean is the mean cardiac output CO in the mean positive airway pressure phase after stabilization and TTcp mean is the mean cardiopulmonary transit time of blood being derived from the envelope of the arterial pulse pressure 5.
Deriving the mean cardiopulmonary transit time of blood TTcp mean by making use of the equations

TTcp mean=t(D)−t(3−2),

or

TTcp mean=t(F)−t(1−2),

wherein t(3−2) is the moment 15 of change from the highest level 10 of PEEP or MPaw PEEP 3 to mean positive airway pressure level or the intermediate level 17 of PEEP or MPaw PEEP 2, t(1−2) is the moment 12 of change from the lowest level 11 of PEEP or MPaw PEEP 1 to average positive airway pressure level or the intermediate level 17 of PEEP or MPaw PEEP 2, t(D) is the time point 16 where the envelope of arterial pulse pressure curve 5 has adapted to intermediate PEEP or MPaw level 17, and t(F) is the time point 14 where the envelope of arterial pulse pressure curve 5 has adapted to intermediate PEEP or MPaw level 17.
Deriving the mean left heart volume LHVmean by making use of the equation

LHVmean=COmean*TTlh,mean,

wherein COmean is the mean cardiac output CO in the mean positive airway pressure phase after stabilization and TTlh,mean is the mean transit time of blood being derived from the envelope of the arterial pulse pressure 5.
Deriving the mean transit time of blood TTlh,mean by making use of the equation

TTlh,mean=t(1−2)−t(E)

wherein t(E) is the time point 13 where envelope curve of arterial pulse pressure 5 starts to rise.
Deriving the slope of Starling curve during TriPAP for the total heart by making use of the difference quotient built from
   delta SV over delta CPBV on 3 PEEP or MPaw levels, and
      for the left heart by making use of the difference quotient built from
delta SV over delta LHV on 3 PEEP or MPaw levels.

1-39. (canceled)

40. A method for using electronic computer processing apparatus to determine a volemic status of a medical patient, the method comprising:
providing the apparatus with data reflecting the patient's arterial pulse pressure;
using the apparatus to determine an arterial pulse pressure envelope from the provided data reflecting the patient's arterial pulse pressure, wherein a value of said arterial pulse pressure envelope is defined for any single heart beat pulse as the difference between the systolic pressure and the diastolic pressure for that given pulse;
estimating the patient's cardiac output; and
using the apparatus to compute an estimate of an internal blood volume of the patient as a product of the patient's estimated cardiac output and an estimated internal blood transit time, wherein said internal blood transit time is estimated by the apparatus based on the determined arterial pulse pressure envelope.

41. The method of claim 40, and further comprising:
providing the apparatus with data reflecting timings of mechanically driven inspiration and expiration of a medical patient undergoing mechanical ventilation;
wherein using the apparatus to compute an estimate of an internal blood volume of the patient includes:
estimating the patient's expiratory cardiopulmonary blood volume as a product of the patient's estimated cardiac output and an estimated cardiopulmonary transit time (CPSVex=CO*TTcp,ex), wherein the cardiopulmonary transit time is estimated as a time difference between a time at which the arterial pulse pressure envelope regains a value equal to or greater than a value it had at the time of the start of a mechanically driven inspiration, and the time of an end of a mechanically driven inspiration (TTcp,ex=t(B)−t(I−E)).

42. The method of claim 40, and further comprising:
providing the apparatus with data reflecting timings of mechanically driven inspiration and expiration of a medical patient undergoing mechanical ventilation;
wherein using the apparatus to compute an estimate of an internal blood volume of the patient includes:
estimating the patient's inspiratory left heart volume as a product of the patient's estimated cardiac output and an estimated inspiratory transit time of blood through the left heart (LHVin=CO*TTlh,in), wherein the inspiratory transit time of blood through the left heart is estimated as a time difference between a time at an end of a mechanically driven expiration, and a time of a beginning of a rise in the arterial pulse pressure envelope (TTlh,in=t(E−I)−t(A)).

43. Electronic apparatus for determining a volemic status of a medical patient, the apparatus comprising computer processor apparatus programmed to:
receive data reflecting the patient's arterial pulse pressure;
determine an arterial pulse pressure envelope from the received arterial pulse pressure data, wherein a value of said arterial pulse pressure envelope is defined for any single heart beat pulse as the difference between the systolic pressure and the diastolic pressure for that given pulse;
receive data reflecting the patient's cardiac output;
compute an estimate of an internal blood volume of the patient as a product of the patient's cardiac output and an estimated internal blood transit time, wherein said internal blood transit time is estimated by the processor apparatus based on the determined arterial pulse pressure envelope.

44. The electronic apparatus of claim 43, wherein the computer processor apparatus is further programmed to:
receive data reflecting timings of mechanically driven inspiration and expiration of a medical patient undergoing mechanical ventilation; and
wherein computing the estimate of the internal blood volume of the patient includes estimating the patient's expiratory cardiopulmonary blood volume as a product of the patient's estimated cardiac output and an estimated cardiopulmonary transit time (CPBVex=CO*TTcp,ex), wherein the cardiopulmonary transit time is estimated by the computer processor apparatus as a time difference between a time at which the arterial pulse pressure envelope regains a value equal to or greater than a value it had at the time of the start of a mechanically driven inspiration, and the time of an end of a mechanically driven inspiration (TTcp,ex=t(B)−t(I−E)).

45. The electronic apparatus of claim 43, wherein the computer processor apparatus is further programmed to:
receive data reflecting timings of mechanically driven inspiration and expiration of a medical patient undergoing mechanical ventilation; and
wherein computing the estimate of the internal blood volume of the patient includes estimating the patient's inspiratory left heart volume as a product of the patient's estimated cardiac output and an estimated inspiratory transit time of blood through the left heart (LHVin=CO*TTlh,in), wherein the inspiratory transit time of blood through the left heart is estimated by the computer processor apparatus as a time difference between a time at an end of a mechanically driven expiration, and a time of a beginning of a rise in the arterial pulse pressure envelope (TTlh,in=t(E−I)−t(A)).
